(19) United States
(12) Patent Application Publication
(10) Pub. No.: US 2019/0336328 A1
(43) Pub. Date: Nov. 7, 2019

(54) FORCED-AIR WARMING BLANKET

(71) Applicant: 3M INNOVATIVE PROPERTIES COMPANY, SAINT PAUL, MN (US)

(72) Inventors: ANDREW J. MCGREGOR, MINNEAPOLIS, MN (US); GLENN R. MAHARAJ, MINNEAPOLIS, MN (US); MATTHEW T. SCHOLZ, WOODBURY, MN (US); WALDO L. LOPEZ, HARRIS, MN (US); GREGG A. PATNODE, WOODBURY, MN (US); JEFFREY O. EMSLANDER, STILLWATER, MN (US)

(73) Assignee: 3M INNOVATIVE PROPERTIES COMPANY, SAINT PAUL, MN (US)

(21) Appl. No.: 16/342,304

(22) PCT Filed: Oct. 18, 2017
(86) PCT No.: PCT/US2017/057079

§ 371 Date: Apr. 16, 2019

Related U.S. Application Data
(60) Provisional application No. 62/411,243, filed on Oct. 21, 2016.

(51) Int. Cl. A61F 7/00; A47G 9/02

(52) U.S. Cl. CPC A61F 7/0097; A61F 7/0085; A61F 2007/006; A47G 9/0223; A47G 9/0215

(57) ABSTRACT

Described herein is a warming blanket having a structure comprising a first layer of material forming a bottom layer with openings to allow a profusion of air through the bottom layer, a second layer of material forming an upper layer wherein the upper layer is coupled to the bottom layer to form an initial shape of the warming blanket and to form a plurality of interconnected air passageways between the first and second layers of material, wherein at least a portion of the structure is deformable in at least one dimension to reshape the periphery of the warming blanket while maintaining the integrity of the interconnecting air passageways throughout the structure; and wherein the deformable portion of the blanket is deformable by at least a 50% elongation. Examples of materials comprising the deformable portions of the warming blanket include low density polyethylene, metallocene polyethylene, polypropylene, parafilm, and polyurethane.

### TECHNICAL FIELD

The disclosure relates to forced air warming blankets.

### BACKGROUND

Patients who are preparing for, undergoing and recovering from a surgical procedure often require and are under the influence of anesthesia as part of the procedure. Due to the effects of the anesthesia, a patient may become unable to regulate their own core body temperature, a condition known as poikilothermia. Under these conditions, and when for example in an air-conditioned environment such as an operating room or a recovery area in a hospital or in a clinic, the lower air temperature and the need for the patient to be at least partially undressed may lead to the patient becoming hypothermic, wherein the core body temperature of the patient may begin to drop in an unintentional and undesirable manner.

One technique used to prevent hypothermia or other undesirable losses in body temperature of a patient when under the influence of an anesthetic is by the use of forced air warming blanket. The blankets are generally constructed of a series of air passages and interconnected air ways formed between two layers of material. The first layer of material is generally non-porous, and is formed on one side of the blanket, and a second layer that is porous, or that includes distributed air-holes (e.g., perforations), is bonded in some fashion to the first layer of material to form the air passages and/or airways. The blanket is configured to be coupled to a device that warms a flow of air to a predefined temperature range, and then directs that warmed air, using a relatively low pressure, into the air passages and/or airways, often through a flexible tube or duct that may also be formed of a non-porous material. The warm air provided into the air passages and/or airway is expelled though the porous material or out through the distributed holes provided by the second layer of material.

By placing the blanket for example over, underneath or in proximity to at least some portion or portions of the patient, the warmed air may be directed to the patient in a manner that assists the body of the patient in maintaining an acceptable core body temperature. Contact with the blanket itself by a portion or portions of the body of the patient may also help assist the body in maintaining the core body temperature within acceptable limits.

### SUMMARY

In general, techniques are described herein allow a disposable warming blanket, also referred to as inflatable blanket, having an initial shape and configuration that may allow the warming blanket to be placed adjacent to and/or cover a certain portion of a patient's body when the patient is in a first position and orientation. The warming blanket is also structured to facilitate reshaping so that the warming blanket, when reshaped, may be placed adjacent to certain portions of the patient body that are different from the portions of the patient body covered by the initial shape of the blanket or covering the sane patient body portions in a different configuration, e.g. arms out vs. arms at the side. Due to the different sizes of patients, for example the size of a child patient versus an adult patient, and/or because of the variations in the positions a patient may need to be placed during a procedure.

Depending on the particular procedures being performed on the patient, a warming blanket may not exist that adequately covers the patient and provides warming without undue interference with the procedure. Further, a particular procedure may require work to be done first on one portion of the patient, while other portions of the patient may require use of the warming blanket having a first configuration or shape, and the one or more later parts of the procedure may require access to different portions of the patient, wherein the warming blanket would need to be moved or otherwise reconfigured. The particular configuration of the warming blanket used during the first portion of this illustrative example procedure may not be configurable for use during the second or later portions of the procedure, thus requiring use of two or more separate warming blankets, adding to the cost of performing the overall procedure on a patient when using conventional warming blankets.

Systems, devices, and techniques are described herein that allow a forced air warming blanket having an initial configuration and shape, and formed of materials and/or configured in various ways to have an initial configuration and shape to be altered to form one or more additional configurations and/or shapes for the same warming blanket. In various examples, at least a portion of the warming blanket comprises a material, such as one or more layers of film, that are formed from deformable material(s) that allow the warming blanket to be stretched and otherwise deformed in various dimensions, in some instances substantially within a planar direction, while maintaining the integrity of the air passages and airways within the warming blanket. The ability to deform the warming blanket while maintaining the integrity of the air passages and airways within the warming blanket allows a single warming blanket to be configurable to a variety of different shapes and configurations for use in a wide variety of different procedures that require patient warming. In addition, in some embodiments, the ability to reconfigure the warming blanket, including reconfiguring the warming blanket before or after inflation of the warming blanket has occurred, allows a same warming blanket to be used for a variety of different surgical procedures or in a single procedure that requires different warming blanket configurations to be used during different phases of the procedure, thus saving cost.

Various examples described in the present disclosure are directed to a warming blanket for warming a patient, the warming blanket comprising: a structure comprising a first layer of material and a second layer of material, the first layer of material forming a bottom layer of the warming blanket, the bottom layer configured to allow a profusion of air through the bottom layer, and the second layer of material forming an upper layer of the warming blanket, the upper layer coupled to the bottom layer around a periphery of the bottom layer to form an initial shape of the warming blanket and to form an interior space between the first layer of material and the second layer of material comprising a plurality of interconnected air passageways; at least one air inlet coupled to the interconnecting air passageways, the inlet configured to receive a flow of air, and to provide the flow of air to the bottom layer through the interconnected air passageways; wherein at least a portion of the structure is configured to be deformable in at least one dimension in order to reshape the periphery of the warming blanket while maintaining the integrity of the interconnecting air passageways throughout the structure, and wherein the portion of the blanket that is deformable is deformable by at least a 20% elongation.

Other examples described in the present disclosure are directed to a system for warming a patient, the system comprising: a source for generating a flow of air; a warming blanket coupled to the source and configured to receive the flow warmed air from the source, and to distribute the flow of air for dispersion to patient, the warming blanket comprising: a structure comprising a first layer of material and a second layer of material, the first layer of material forming a bottom layer of the warming blanket, the bottom layer comprising openings configured to allow a profusion of air through the bottom layer, and the second layer of material forming an upper layer of the warming blanket, the upper layer coupled to the bottom layer around a periphery of the bottom layer to form an initial shape of the warming blanket and to form an interior space between the first layer of material and the second layer of material comprising a plurality of interconnected air passageways; wherein at least a portion of the structure is configured to be deformable in at least one dimension that is co-planer with a central plane of the warming blanket in order to reshape the periphery of the warming blanket having the initial shape so that the structure remains substantially within an area having a thickness dimension of the initial shape of the warming blanket and while maintaining the integrity of the interconnecting air passageways throughout the structure, and wherein the portion of the blanket that is deformable is deformable by at least a 20% elongation.

Other examples described in the present disclosure are directed to method of reshaping a warming blanket, the method comprising: positioning the warming blanket to form the warming blanket into an initial shape; deforming the warming blanket to form a shape with respect to the periphery that is a different shape from the initial shape formed by the periphery while maintaining the integrity of the passageways providing distribution of air flows through the interior space of the warming blanket, wherein a portion of the blanket that is deformed is deformed by at least a 20% elongation; and inflating, by the source generating the flow of air, the warming blanket to maintain the warming blanket in the shape that is different from the initial shape.

### BRIEF DESCRIPTION OF DRAWINGS

FIG. 1 is illustrative of an example patient warming system including a warming blanket in accordance with one or more example implementations and techniques described in this disclosure.

FIG. 2A illustrates a top and cutaway view of various dimensional aspects and other characteristics of an example warming blanket according to the techniques described in this disclosure.

FIG. 2B illustrates an example warming system including an example of a variation of the warming blanket of FIG. 2A in accordance with one or more example implementations and techniques described in this disclosure.

FIG. 2C illustrates an example warming system including an examples of a variation of the warming blanket of FIG. 2A in accordance with one or more example implementations and techniques described in this disclosure.

FIG. 3A illustrates a top view of the warming blanket of FIG. 2A, deformed to reshape the warming blanket into a shape and configuration that is different from the initial shape and configuration.

FIG. 3B illustrates an example warming system including a warming blanket of FIG. 3A in accordance with one or more example implementations and techniques described in this disclosure.

FIG. 4A illustrates a top view of the warming blanket of FIG. 3A, further deformed to reshape the warming blanket into a shape and configuration that is different from the initial shape and configuration, and that is different from the deformed shape and configuration illustrated in FIG. 3A.

FIG. 4B illustrates an example warming system including a warming blanket of FIG. 4A in accordance with one or more example implementations and techniques described in this disclosure.

FIG. 5 illustrates a top view of an example warming blanket according to the techniques described in this disclosure.

FIG. 6 is an example of a rectangular warming blanket according to the techniques described in this disclosure.

FIG. 7 is an example of an elliptically shaped warming blanket according to the techniques described in this disclosure.

FIG. 8 illustrates a top view of another example warming blanket according to the techniques described in this disclosure.

FIG. 9 illustrates a method according to various examples is accordance with the devices, systems, and techniques described in this disclosure.

The drawings and the description provided herein illustrate and describe various examples of the inventive methods, devices, and systems of the present disclosure. However, the methods, devices, and systems of the present disclosure are not limited to the specific examples as illustrated and described herein, and other examples and variations of the methods, devices, and systems of the present disclosure, as would be understood by one of ordinary skill in the art, are contemplated as being within the scope of the present application. In addition, one or more reference numbers may be first introduced in a figure of the application to refer to a device, a method step, or some other aspect related to the figure, wherein the same reference number may then be used in a subsequent figure or figures to refer to the same device, method step, or other aspect as described with respect to the original figure, but without a particular reference to the same reference numbers in the description corresponding to the subsequent figure(s). In such instances and unless stated otherwise, the reference numbers as used in the subsequent figure or figures incorporate all of the features, functions, and the equivalents thereof of the devices, method steps, or other aspects described with respect to the reference number where first introduced and described.

### DETAILED DESCRIPTION

As discussed above, systems, devices, and techniques are described herein with respect to a forced air warming blanket having an initial configuration and shape, and formed of materials and/or configured in various ways that allow the initial configuration and shape to be altered to form one or more additional configurations and/or shapes for a warming blanket. Although examples of the systems, devices and techniques described throughout this disclosure refer to forced air warming blankets, these systems, devices, and techniques are not necessarily limited to forced air warming blankets, and may be equally applicable to warming pad, warming tubes, and other patient warming devices, and the equivalents thereof, as would be understood by one or ordinary skill in the art.

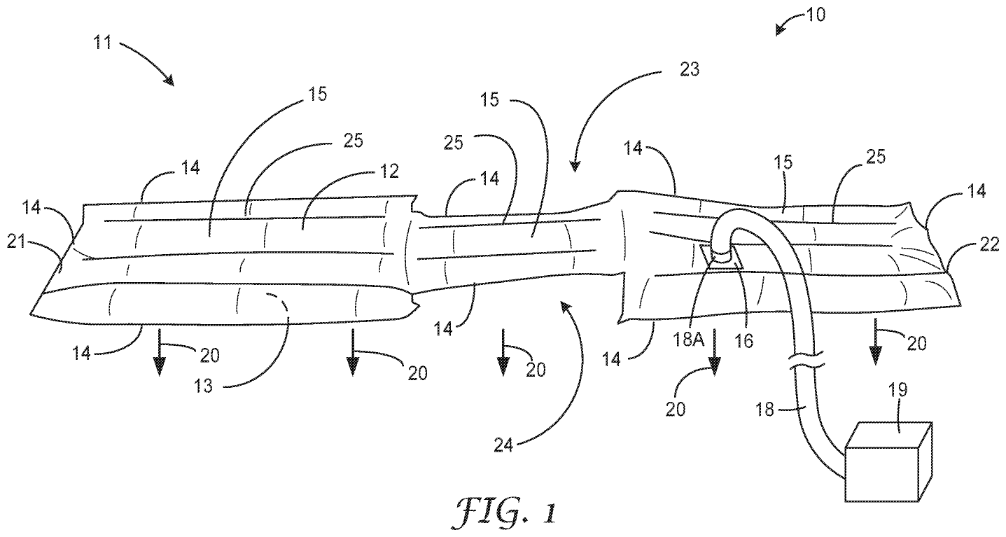

FIG. 1 is an illustrative of an example warming system 10 that includes a warming blanket 11 in accordance with one or more example implementations and techniques described in this disclosure. In this example, warming blanket 11 includes upper layer 12 having a sheet of material, and a bottom layer 13 having a sheet of material. In various examples, upper layer 12 is a separate sheet of material that is bonded to bottom layer 13 along a periphery 14 of each the layers 12, 13. In other examples, upper layer 12 is a same sheet of material folded over bottom layer 13, or formed as a tube, and then bonded (sealed) along portions of the periphery 14, such as at the end 21 and the end 22, where the material is not already a continuous sheet, or along cutouts, such as cutout 23 and cutout 24 formed along the periphery 14 in each of the upper layer 12 and the bottom layer 13. Upper layer 12 may also be bonded to bottom layer 13 at portions of the upper layer 12 not along periphery 14, to form pleats 25 where upper layer 12 contacts bottom layer 13. The pleats 25 may not extend to the periphery 14 along the edges of warming blanket 14, and the spaces between upper layer 12 and bottom layer 13 and between the pleats 25 form a plurality of interconnected passageways, generally indicated as passageways 15.

Passageway 15 within warming blanket 11 are coupled to an inlet 16 including an opening to the passageways from outside the warming blanket 11, in some examples to receive a coupling 18A. Coupling 18A may also couple inlet 16 to a tubular air hose 18 at one end of hose 18, the opposite end of hose 18 coupled to a source 19 for a flow of air. Inlet 16 may include a collar 16A surrounding the opening in 16A that may form a ring or thicker portion of inlet 16 to allow for coupling and securing a device, such as couplie 18A, to inlet 16. In various examples, warming blanket 11 further comprises an opening 17, which may be located on bottom layer 13, and including another opening to the passageways 15 from outside the warming blanket 11. Opening 17 may be configured in a same manner as inlet 16. Opening 17 may in some examples be located on upper layer 12. Opening 17 may initially be sealed in some manner to seal the opening provided through opening 17 in order to block the flow of air from passageways 15 through opening 17, and may be unsealed, for example by removing a seal (not shown in FIG. 1) from opening 17 to allow the warming blanket 11 to be coupled to another warming blanket (not shown in FIG. 1).

Source 19 may be any device that is configured to warm a flow of air to a temperature that may be applied to a patient (not shown in FIG. 1) safely while the patient is preparing for, undergoing, and/or recovering from a procedure where the patient is under the influence of an anesthetic or to otherwise warm a patient that may feel cold. The warm air from source 19 is provided to hose 18 at a relatively low pressure, for example a pressure less than 100 mm Hg, and in some examples less than 10 mm HG, and flows through hose 18 to inlet 16, where the air flow continues into the passageways 15 of warming blanket 11, and inflating the warming blanket to fill the passageways 15 via the low pressure air flow provided to inlet 16. In some embodiments more than one inlet may be provided. Throughout the disclosure, the flow air provided to inflate one or more of the warming blankets may be described as a “flow of warmed air.” In reference to a flow of air or a flow of warmed air, the air provided to the warming blanket or warming blankets may be warmed to a temperature of about 36 to 43 degrees C. (Celsius). However, a flow of air provided to a warming blanket as described in this disclosure also includes provided a flow of air at some other temperature, for example at an ambient temperature, or air that has been cooled to below an ambient temperature. In some examples, source 19 provides pressurized air at a flow rate of 40 to 50 cubic feet per minute (CFM). In some examples, source 19 provides pressurized air at a flow rate of 35 to 60 CFM. In some examples, source 19 provides pressurized air at a flow rate of 40 to 60 CFM. In some cases, source 19 provides pressurized air at a flow rate of 43 to 47 CFM.

Each of the upper layer 12 and the bottom layer 13 may include one or more sheets, where each sheet may be formed from a different material. In some implementations, the upper layer 12 and/or the bottom layer 13 may include an underside sheet formed from a flexible, fibrous, preferably non-woven structure composed of polymeric materials capable of bonding to an upper side sheet of a heat-sealable polymeric material. For example, the underside sheet may be a non-woven, hydroentangled polyester material and the upper side layer may include a polyolefin such as a polypropylene film which is extrusion-coated, thermally laminated, or adhesively laminated onto the polyester layer. Alternatively, the underside sheet may comprise a non-woven, paper-based material to which the upper side layer, including either a polyethylene or polypropylene film, has been glue laminated. In one embodiment, the upper side and underside sheets can be made with a stratum of absorbent tissue paper prelaminated with a layer of heat-sealable plastic. In some cases, both the first layer and the second layer can include a same polymer material.

In some embodiments, the bottom layer 13 includes the upper side sheet and the underside sheet, and the upper layer 12 comprises the same material as the upper side sheet of the second layer. The upper layer 13 thus may include a sheet of plastic bonded to the plastic upper side of the second layer. It is preferably attached by a continuously-running web process including stations that provide an interruptible heat-sealing process. This interruptible heat sealing process can be controlled to form elongated heat seals, shown as pleats 25, that define the inflatable channels therebetween. The seals can be formed as continuous air impervious seals or discontinuous air permeable seals. The interruptible heat sealing process can be used to form the continuous seams, one of which is the periphery 14 at the peripheral of the top layer 12 and the bottom layer 13. In some cases, the interruptible heat sealing process can be used to form the discontinuous heat seals. In some cases, absorbent material can be applied to the warming blanket 11, for example, applied as a single material layer. The absorbent material can be bonded to the upper plastic layer by heat processing or by adhesive bonding.

In some embodiments, the warming blanket 11 is enabled to bathe a patient in the thermally controlled inflation medium introduced into the warming blanket 11 when inflated, via an air permeable layer, the first layer and/or the second layer. A layer can be air permeable using various materials or mechanical structures, for example, air-permeable materials, apertures, interstices, slits, or the like. In some implementations of an air permeable sheet with apertures, the density of apertures can vary among areas and/or inflatable sections.

In some embodiments, the upper layer 12 and/or the bottom layer 13 are made from a polyolefin non-woven extrusion coated, each with a coating of polypropylene on one side. In some other embodiments, the upper layer 12 and/or the bottom layer 13 can be poly lactic acid spunbond with polyolefin based extrusion coat. One of the upper layer 12 and bottom layer 13 may have holes formed by punching, slitting, or cutting to permit the flow of pressurized inflation medium from the inflated section through the layer. In some cases, the holes can be opened through both layers. In some cases, when the warming blanket 11 is assembled, the polypropylene-coated side of the upper layer 12 is sealed to the polypropylene-coated side of the bottom layer at the periphery 14, and at the one or more locations such as pleats 25 to form the construction. The sealing process can use various techniques, for example, ultrasonic welding, radio frequency welding, heat sealing, or the like. Alternatively, the upper layer 12 and bottom layer 13 may each include a laminate of polypropylene and polyolefin web with holes formed in at least one of the layers to support passage of pressurized air. In yet another embodiment, at least one of the layers can use air permeable material, for example, spunbond-meltblown-spunbond (SMS) nonwoven material, or the like.

Upper layer 12 of the warming blanket 11 is generally comprised of a material that may be formed from a porous or a non-porous material that may or may not be perforated. If made from a porous material or from a non-porous material, the upper layer 12 may provide a path of air flow from passageways 15 through upper layer 12 for some portion of the flow of air within passageways 15 to exit the warming blanket. If upper layer is formed from a non-porous material that is not also perforated, upper layer 12 does not provide a path for air to flow from passageways 15 through the upper layer 12. In one preferred embodiment, the non-porous and non-perforated characteristic of the upper layer 12 helps to maintain a low level of air pressure within the passageways 15 based on the air flow and air pressure provided by source 19 to inlet 16. In contrast, bottom layer 13 is porous, either by virtue of being formed from a porous material, such as a woven, knit, or nonwoven fabric material, or by being formed from a non-porous or porous material that has been further processed to include a plurality of perforations (e.g., through-holes in bottom layer 13, not specifically shown in FIG. 1) that allow air to flow from passageways 15 to an area outside the warming blanket, the airflow generally indicated by arrows 20 in FIG. 1. In some embodiments both upper layer 12 and lower layer 13 may be porous or otherwise have perforations, such as openings or slits, through which the air may flow out of the patient warming blanket.

In instances where bottom layer 13 is a porous material, the air flow generally indicated by arrow 20 will be distributed across most of the surface area including the bottom surface 13, wherein the porosity and the surface area of bottom layer 13 are configured to allow enough backpressure for inflation of passageways 15, and thus to provide a gentle and warming air flow when source 19 is providing an air flow to inlet 16 within a predetermined range of pressures and volumetric rates of air flow. In instances were bottom layer 13 is a non-porous material but has been further processed to include perforations (e.g., through-holes) extending through the material forming the bottom layer 13, the perforations may be sized and distributed over the surface areas of the bottom layer 13. The sizing and distributing of the perforations is configured to allow the air flow, generally indicated by arrows 20, to be provided across substantially most of surface area including the bottom layer 13, while providing enough backpressure and to allow for inflation of passageways 15, and thus to provide a gentle and warming air flow (e.g., arrows 20) when source 19 is providing the air flow to inlet 16 within a predetermined range of pressures and rates of air flow.

Warming blanket 11 may be provided in and initial configuration relative to the shape of the periphery of the blanket. Prior to inflation of the blanket, the blanket is deformed to a shape that is different from the initial shape of the periphery of the blanket. Warming blanket 11 may then be placed over a patient (not shown in FIG. 1) and proximate to portions of the body of the patient, so that the bottom layer 13 is facing the portions of the patient that are to be warmed, and subsequently inflated. For example, warming blanket 11 may be place over the upper torso and arms of a patient, for example during times when direct or immediate access to these portions of the patient are not required by other personnel, such as a physician or a surgeon. In other examples, the warming blanket may be detachably made as part of a gown (not shown in FIG. 1) that may be worn by the patient while waiting for the process that is to be performed on the patient to begin. In such instances, the detachable warming blanket may be detached from the gown when the gown is fully or partially removed from the patient in preparation for the actual procedure, and the warming blanket repositioned proximate to the patient to provide patient warming. Once in place, the air flow from the warming blanket 11 (generally indicated by arrows 20) may be directed to the portions of the patient proximate to the warming blanket, and thus provide a gentle and warming air flow and or a warm surface provided by the external surface of bottom layer 13 that warms the patient.

As illustrated in FIG. 1, warming blanket 11, when inflated by the air flow provided by source 19, provides a particular shape dictated to a large degree by the periphery 14, and by pleats 25, which hold the upper layer 12 and the bottom layer 13 in relative close proximity to one another across the length and width dimensions of the warming blanket. For example, the warming blanket 11 as illustrated in FIG. 1 provide a substantially rectangular shape relative to the length dimension (e.g. between ends 21 and 22), and the width dimension, (dimension perpendicular to and coplanar with the length dimension), but also including cutouts 23 and 24 that narrow the width dimension of the warming blanket over a central portion of the warming blanket. This initial configuration and shape for warming blanket 11 may be used to warm the upper torso and outstretched arms of a patient for example when the patient is lying on their front or back side with arms positioned in an outstretched direction perpendicular to the torso. In such instances, the narrow portion of the warming blanket created by cutouts 23, 24 may be placed over the torso, and the portions of the warming blanket extending outward from this central portion may be used to cover and warm each arm, respectively, of the patient.

However, this particular shape may not be effective to providing warming for a patient when the patient is required to be in some position different from the positions described above, or for example where access to the upper torso and/or an arm of the patient is required as part of the procedure being performed on the patient. As described above, this may require for example a hospital or a clinic to stock a variety of different warming blankets that may be required for different procedures, thus adding to inventory costs. In addition, certain procedures may require multiple different patient positions that may not be accommodated by use of a single conventional warming blanking having for example a relatively fixed initial configuration and shape when inflated. In these instances, multiple warming blankets may be required in order to complete the procedure being performed on the patient, again adding to the overall cost of the procedure.

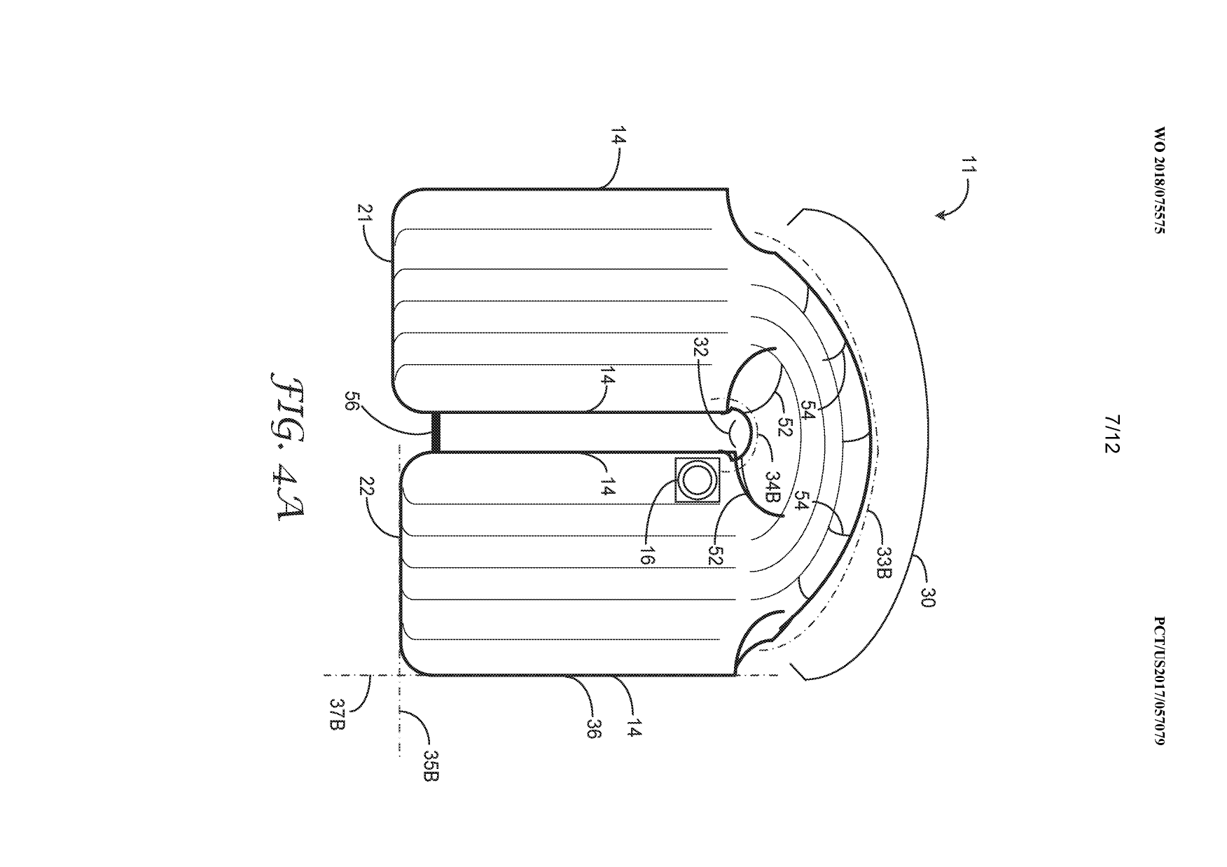

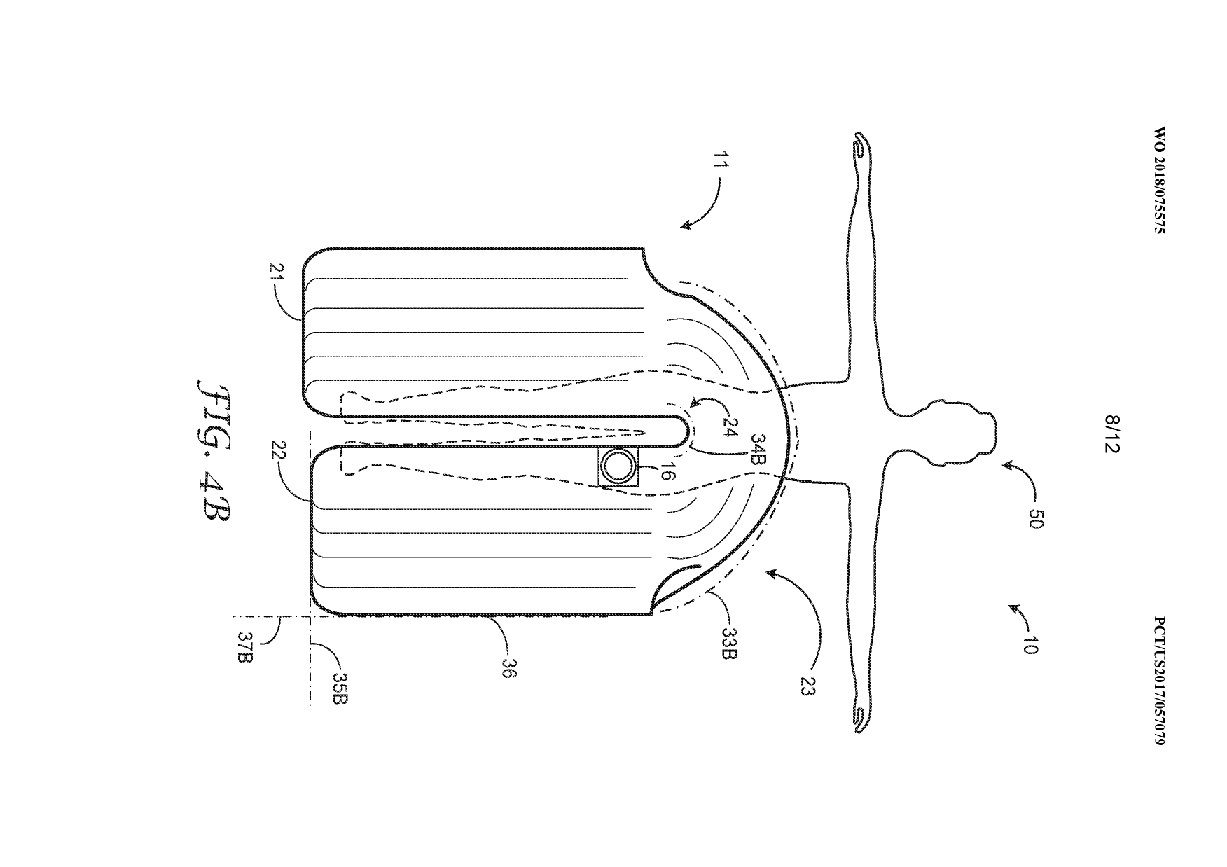

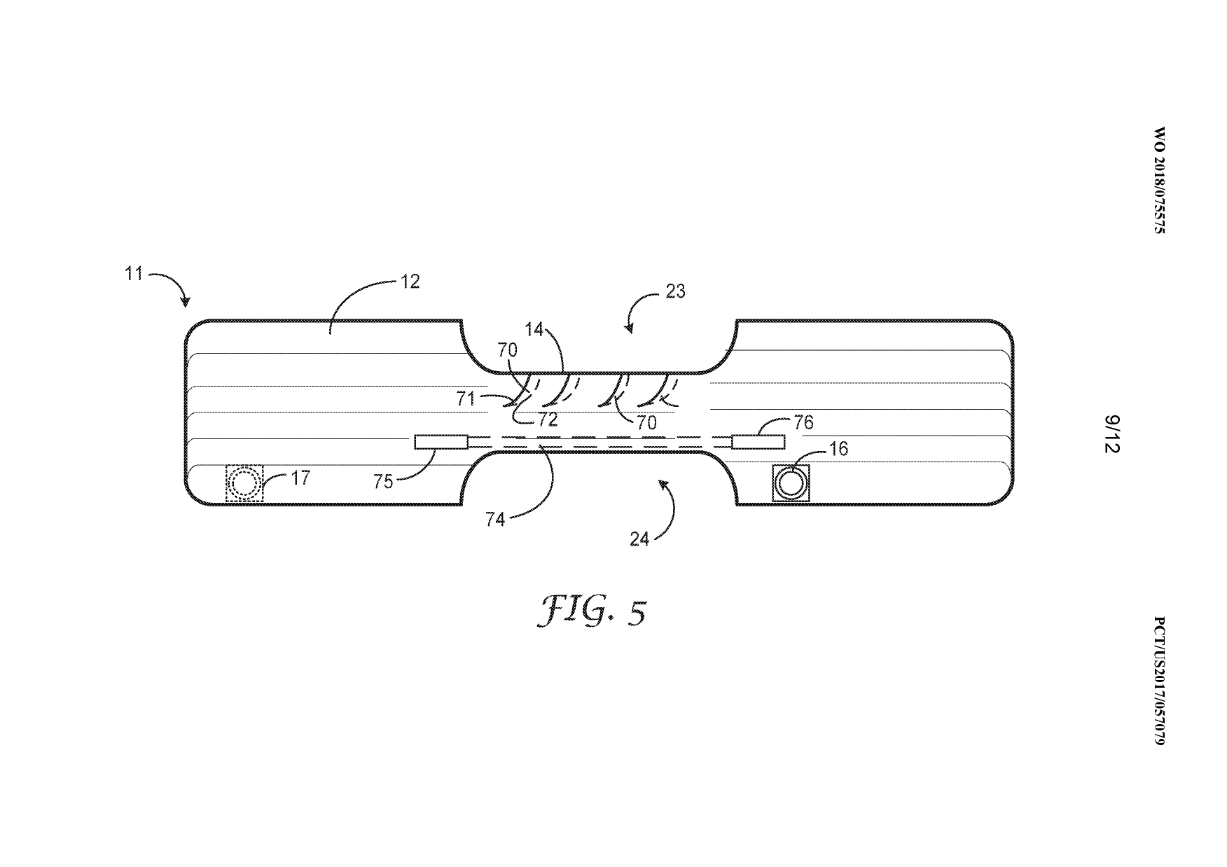

As further described below, examples of warming blanket 11, and the equivalents and variations thereof, have at least a portion of the warming blanket that comprise a material or materials that allow the warming blanket to be deformed, for examples stretched along various dimensions corresponding to generally planar dimensions, in order to reshape the warming blanket into a shape that is different from the initial shape and configuration of the warming blanket prior to inflation of the blanket. However, in various examples, deformation of the warming blanket can occur either before or after the warming blanket is inflated with a flow of air, such as a flow of air provided at inlet 16 by source 19. In various examples, deformation of the warming blanket to reshape the warming blanket includes plastic deformation of at least apportion of the warming blanket such that once deformed, the warming blanket tends to maintain the shape that the blanket was reformed to take on. In other examples, deformation of the warming blanket to reshape the warming blanket includes elastic deformation of a least a portion of the warming blanket, such that once elastically deformed, the portion of the warming blanket deformed may be returned to substantially the initial configuration of the warming blanket before the warming blanket was elastically deformed. In various examples, the warming blanket includes one or more securing ties (not shown in FIG. 1, but for example securing tie 56 as shown and described with respect to FIG. 4A, and securing tie 74 as shown and described with respect to FIG. 5), which help maintain the deformed warming blanket in the reconfigured shape once the blanket has been deformed.

In various examples, the material or materials that comprise the portions or portions of the warming blanket that are deformable include materials that can be formed as films used to form upper layer 12, bottom layer 13, both layers 13, 14, and/or any portions thereof. Examples of materials that deform include very low density polyolefins, low density polyethylene, linear low density polyethylene, polypropylene, and olefin copolymers such as ethylene-vinyl acetate (EVA). A preferred plastically deforming material would be very low density polyethylene optionally containing fillers. Examples are metallocene polyolefin and parafilm. Examples of materials that are elastic are materials that include polyolefins, such as metallocene polyethylenes such as Engage® polyethylenes (commercially available from Dow Chemical Company, Midland Mich.), polyurethanes such as polyester or polyether polyurethanes (e.g., “Estane® thermoplastic polyurethane,” commercially available from B. F. Goodrich, Cleveland Ohio), polyesters such as polyether polyester (e.g., “Hytrel® polyester elastomer,” commercially available from Du Pont Co., Wilmington, Del.), and polyamides such as polyether polyamides (e.g., “Pebax® Resins” commercially available from ELF Atochem, North America, Inc., Philadelphia, Pa.) and acrylic block copolymers such as Kurarity block polyacrylates available from Kuraray America, Houston Tex.

In various examples, the material forming the portion of blanket or the blanket itself that is deformable allows the material to be deformed by an elongation of at least 20% the blanket or of the deformable portion of the blanket. In some examples, the material forming the portion of blanket or the blanket itself that is deformable allows the material to be deformed by an elongation of at least 30% the blanket or of the deformable portion of the blanket. In other examples, the material forming the portion of blanket or the blanket itself that is deformable allows the material to be deformed by an elongation of at least 40% the blanket or of the deformable portion of the blanket. In other examples, the material forming the portion of blanket or the blanket itself that is deformable allows the material to be deformed by an elongation of at least 50% the blanket or of the deformable portion of the blanket.

In various examples, the warming blanket is configured so that when a force of deformation applied is less than 25 Newtons or even less than 10 Newtons at 25% strain for a test sample of the deformable portion of the blanket that is 2.54 cm wide, according to a tensile strength testing with a gauge length of 50 millimeters (mm) and cross-head speed (pull speed) of 254 millimeters (mm) per minute. In at least one embodiment, % elongation can refer to the elongation at the Fmax, the maximum applied load, which can differ between various materials. The Fmax can occur at or around the elastic limit of the material being tested. In at least one embodiment, the % elongation of a deformable material can be determined using ISO 9073-3 (1989) (e.g., at room temperature).

In at least one embodiment, the % elongation to force applied ratio of a deformable material can be at least 1.25 to 1, at least 1.5 to 1, at least 10 to 1, at least 20 to 1, or even at least 30 to 1. In at least one embodiment, the force applied can be Fmax.

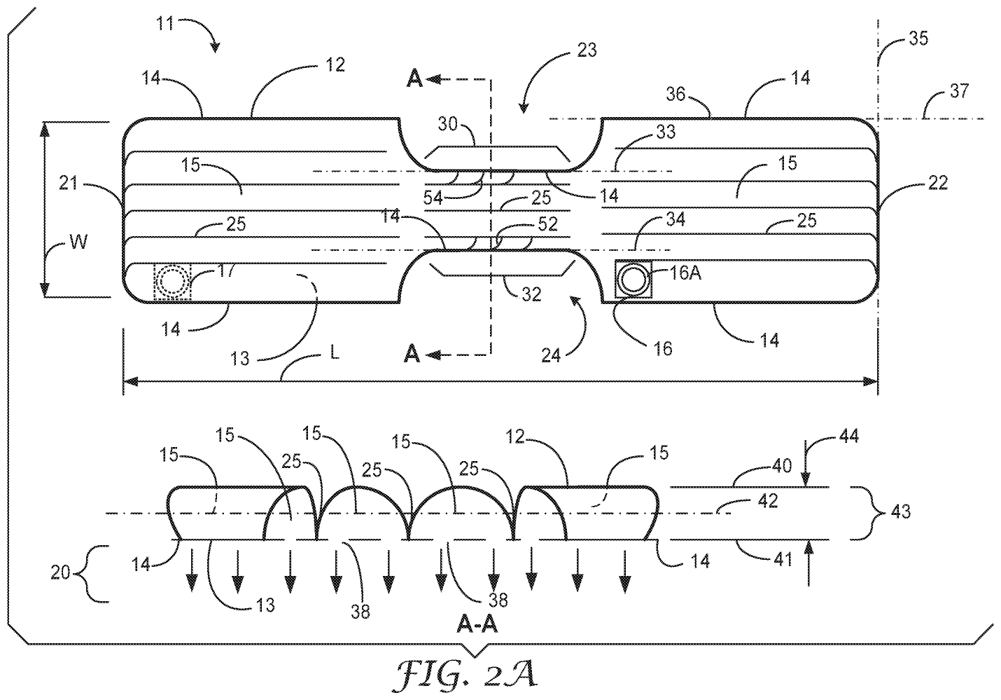

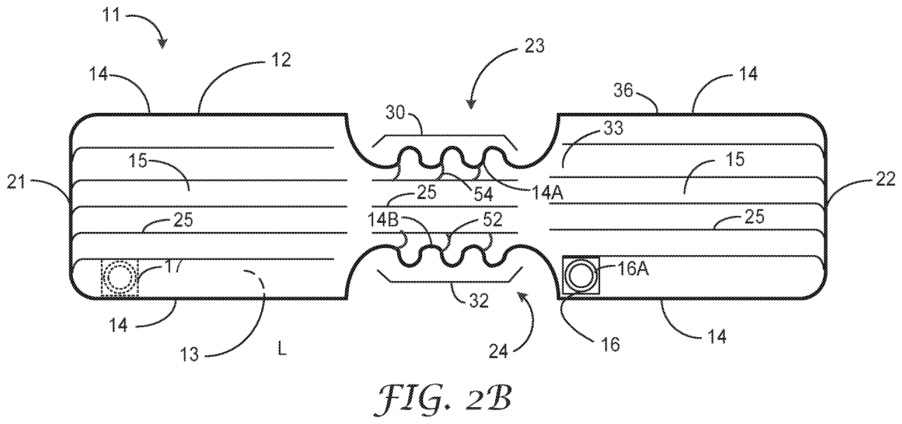

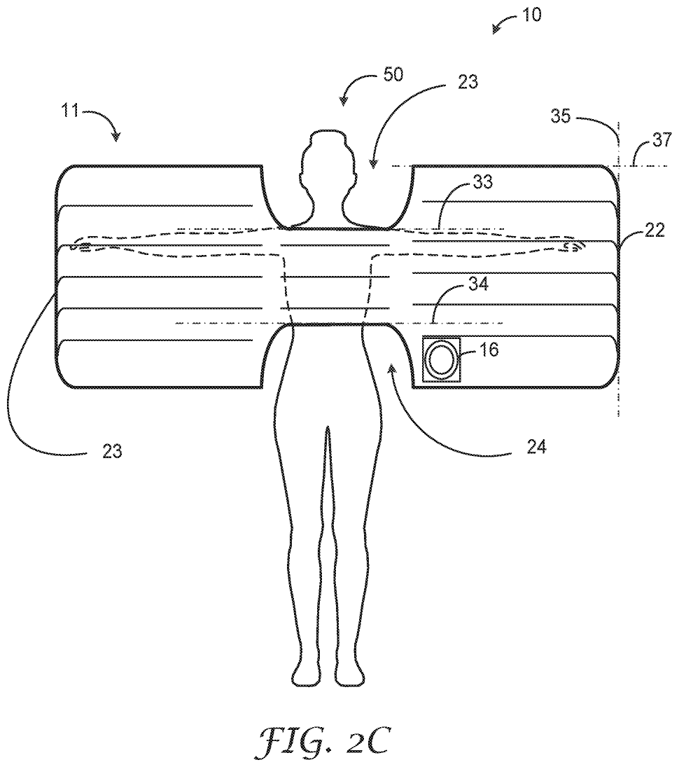

FIG. 2A illustrates a top and cutaway view of various dimensional aspects and other characteristics of an example warming blanket 11 according to the techniques described in this disclosure. As illustrated, warming blanket 11 includes the features of warming blanket 11 illustrated and described with respect to FIG. 1, including a periphery 14 coupling upper layer 12 and bottom layer 13, and providing an initial shape, including a width dimension W, and a longitudinal dimension L between ends 21 and 22, and a narrowed central portion midway along this longitudinal dimension, generally indicated by cutouts 23, 24. Longitudinal dimension L is some examples is in a range of 60 to 90 inches, and width dimension W may be in a range of 10 to 40 inches. Upper layer 12 and bottom layer 13 form passageways 15 that are coupled to receive a flow of air provided to inlet 16, and to distribute the air throughout passageways 15 to be expelled out through bottom layer 13.

FIG. 2A includes a cutaway view A-A showing a view of warming blanket 11 looking into the central portion of the warming blanket toward end 21. As shown in view A-A, upper layer 12 is sealed or otherwise in contact and bonded with bottom layer 13 at the periphery 14, and also at pleats 25, to form passageways 15 between upper layer 12 and bottom layer 13. Bottom layer 13 includes a porous material, or may be a perforated non-porous material, having passages or through-holes, generally indicated as perforations 38, that allow a flow of air, generally indicated by arrows 20) to exit passageways 15 through bottom layer 13 when warming blanket 11 is provided a flow of air to passageways 15. Bottom layer 13 is generally a sheet of material having a planar configuration when lying on a horizontal rigid surface within periphery 14, generally coplanar with plane 41, and upper layer 12 is generally a sheet of material, having ridges formed by passageways 15, but generally having peaks falling with a planar area indicated by plane 40. While shown as relatively uniform size, pleats 25 also may vary in width and height such that their peaks do not fall largely in the same plane. However, bottom layer 13 is not limited to having a substantially flat planar configuration, and may have some variations, such as for example having ridges formed by passageways 15, and for example variations created by pleats 25, in a similar manner described for upper layer 12. The upper layer 12 and the bottom layer 13 are generally contained within an area between planes 40 and 41, generally indicated by area 43, and having a thickness dimension 44. In various examples of warming blanket, thickness dimension 44 having an inflated thickness value in a range between 3 and 15 inches.

In various examples, a central plane 42 can be illustratively constructed midway between planes 40 and 41 relative to the thickness dimension 44 of warming blanket 11. As further illustrated and described below, deformation of the warming blanket 11 to reshape the warming blanket may include deforming the blanket in a dimension that is generally coplanar with central plane 42, allowing the areas between planes 40 and 41 to not increase or decrease by more than 50% and preferably by not more than 25% in the same dimension of inflated thickness dimension 44, after deforming or reshaping the warming blanket with respect of the shape of periphery 14 to some extent, and while maintaining the integrity of the passageways 15 through the warming blanket. By “maintain the integrity of passageways” 15 it is meant that the entire blanket still inflates and preferably does so in less than 30 seconds, more preferably in less than 20 seconds and most preferably in less than 10 seconds when using a forced air blower at a pressure of 100 mmHg or less and a flow rate of 40 to 50 cubic feet per minute (CFM). In other words, deforming the warming blanket to reshape the periphery of the warming blanket while maintaining thickness dimension of +/−50% across the upper and bottom layers of the warming blanket may also not restrict the flow of air to the passageways 15, for example by kinking, crushing, or otherwise obstruction the passageways 15 and/or the airways coupling in the passageways to inlet 16 across substantially the entirety of the bottom surface 13 of the warming blanket. When deformed, the warming blanket 11 is configured to maintain the integrity of the passageways 15, and thus continue to be able to deliver substantially the same air flow (represented by arrows 20 in view A-A) in the deformed shape as would have been available when the warming blanket was in the un-deformed initial shape and configuration.

Referring again to FIG. 2A, warming blanket 11 includes a dimension 30 along the periphery 14 within cutout 23, and a dimension 32 along the periphery 14 within cutout 24. As shown in FIG. 2A, a typical dimension for dimension inset 30 and dimension inset 32 when warming blanket is in the initial configuration, as illustrated in FIG. 2A, is in a range of about 12 to 24 inches. In various examples, at least the central portion warming blanket 11, generally indicated by the portion of warming blanket included between dimension inset 30 and dimension inset 32, includes a material or materials that are deformable to allow periphery 14 to be stretched or otherwise reshaped, while maintaining thickness dimension 44 relative to the central plane 42 of the warming blanket to +/−50% of the original dimension, and while maintain the integrity of the passageways 15 throughout the warming blanket, including the portions of the passageways included in the central portion of the warming blanket. In various examples, pleats 54 may be provided along a portion of cutout 23 to allow expansion of upper layer 12, to aid in expanding dimension 30 along the outside curve of periphery 14 within cutout 23. Similar pleats may also be included on bottom layer 13. In addition, pleats 52 may be provided along a portion of cutout 24 to allow folding of upper layer 12, to aid in lessening dimension 32 along the inside curve of periphery 14 within cutout 24. Similar pleats may also be included on bottom layer 13.

As further described below, warming blanket 11 in the initial configuration including various axes, such as axes 33, 34, 35, and 37, that have an initial orientation when warming blanket is in the initial shape and configuration shown in FIG. 2A. For example, axis 33 includes an axis that aligns with the longitudinal dimension of the warming blanket 11 along periphery 14 and inside cutout 23, and axis 34 includes an axis that aligns with the longitudinal dimension of the warming blanket 11 along periphery 14 and inside cutout 24. Axis 35 includes an axis that aligns with the width dimension of the warming blanket 11 along periphery 14 at end 22, and axis 37 includes an axis that aligns with the longitudinal dimension of the warming blanket 11 along periphery 14 and along a side 36 of periphery 14 outside cutout 23.

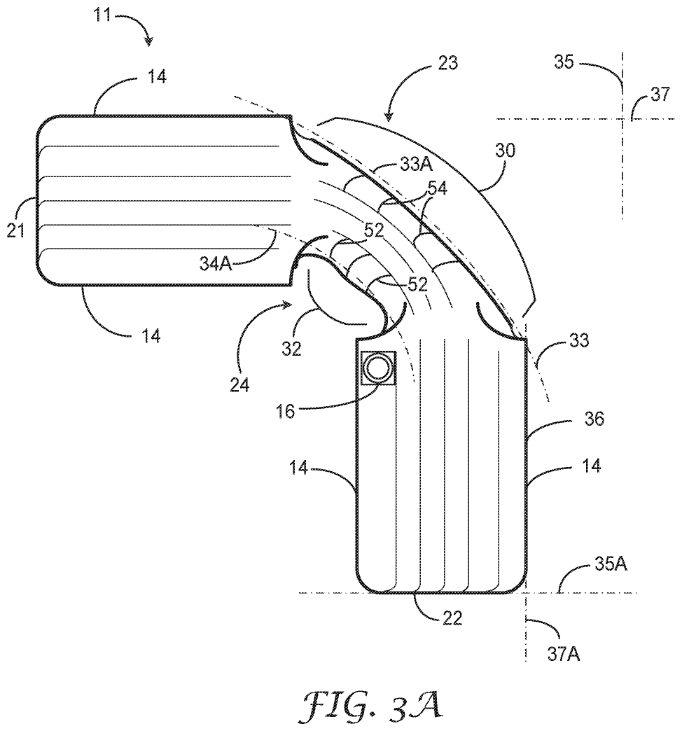

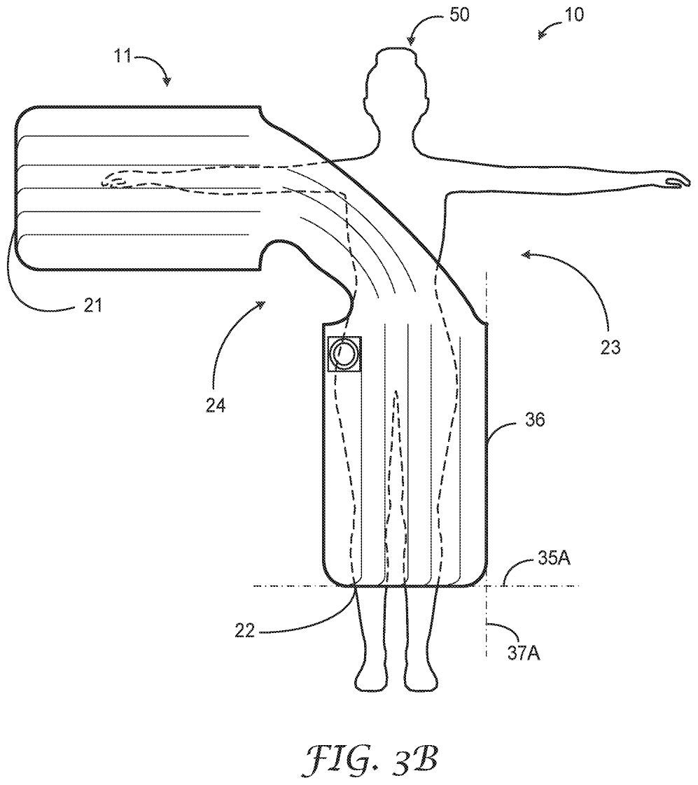

Each of these axes lie in a plane that is coplanar with or in a plane that is parallel to the central plane 42 of warming blanket 11 when lying on a rigid horizontal surface, and has an initial orientation (e.g., axis direction) as shown in FIG. 2A. As illustrated and described with respect to FIGS. 3A-3B and 4A-4B, the orientation of one or more of these axes may be changed in a direction and/or in a dimension in order to reshape the periphery 14 of the warming blanket 11, while the axes remain approximately coplanar or in a plane parallel to central plane 42 of the warming blanket, and while maintaining the integrity of the passageways 15. As would be understood by one of ordinary skill in the art, many other illustrative axes could be associated with the configuration of warming blanket 11 as shown in FIG. 2A, and are contemplated by the examples described herein.

FIG. 2B illustrates an example of a variation of the warming blanket of FIG. 2A in accordance with one or more example implementations and techniques described in this disclosure. Warming blanket 11 as shown in FIG. 2B may include any combination of the features shown and described above with respect to warming blanket 11A or the equivalents thereof, with the variations as described below. As shown in FIG. 2B, the periphery warming blanket 11 within the cutout area 23 is formed to have a wavy or sinusoidal shape, as indicated by periphery 14A. In addition, the periphery of warming blanket 11 as shown in FIG. 2A may have a way or sinusoidal shape as indicated by periphery 14B. The shape of periphery 14A and 14B are limited to a particular shape, such as a sinusoidal shape, and may be any shape having a linear distance that when traced along periphery 14A, and/or 14B, have a linear distance that is greater than the linear distance for a straight line. In various examples, only periphery 14A or 14B is provided as a wavy or sinusoidal (e.g., non-linear) shape.

By providing the one or both of periphery 14A, 14B as a wavy, sinusoidal, or some other non-linear shape, the section including the periphery 14A, 14B provides a slack to the dimension 30 and or 32 to allow for stretching of that section of periphery. When periphery 14A or 14B is provided on as a periphery on a side of the warming blanket 11 that is opposite a side being stretched, the periphery 14A or 14B promotes controlled bending of that portion of the periphery. Either or both of periphery 14A, 14B may be provided in conjunction with pleats 54, 52, respectively, to further aid in and control the bending of warming blanket 11 in the portion of warming blanket 11 where at least one of periphery 14A, 14B are provided.

FIG. 2C illustrates an example warming system 10 including a forced air warming blanket 11 of FIG. 2A or FIG. 2B in accordance with one or more example implementations and techniques described in this disclosure. As illustrated, warming blanket 11 is shown in an initial configuration as illustrated and described for example with respect to FIG. 2A or FIG. 2B, located proximate to the upper torso and arms of a patient 50, and configured to receive a flow of warmed (or cooled) air at inlet 16 from a source, such as source 19 and through a hose such as hose 18 as shown in FIG. 1. As illustrated in FIG. 2C, axes 33, 34, 35, and 36 remain in a same orientation as illustrated and described above with respect to FIG. 2A.

FIG. 3A illustrates a top view of the warming blanket 11 of FIG. 2A or 2B, deformed to reshape the warming blanket into a shape and configuration that is different from the initial shape and configuration. As illustrated, warming blanket 11 has been deformed so that the dimension indicated by dimension bracket 30 has been stretched to increase this dimension associated the periphery 14 within cutout 23, and the axis 33 along this same portion of periphery 14 has been re-oriented from a straight line to form an arc shape, illustrated as axis 33A. In addition, the axis 34 that was originally a straight line along periphery 14 within cutout 24 is also re-oriented, may include for example in some embodiments an arc shape as illustrated by axis 34A, and the dimension associated with dimension bracket 32 is compressed to have a smaller dimensional value. As illustrated in FIG. 3A, axis 35, that originally aligned with end 22 of the warming blanket has been re-oriented approximately 90-degrees, as represented by axis 35A. Similarly, axis 37, which originally aligned with side 36 of warming blanket 11, is re-oriented approximately 90 degrees, as represented by axis 37A. In all instance of re-orientation of axes 33, 34, 35, and 37, the direction and dimension associated with the re-orientation of these axes is coplanar or is contained within a plane that is parallel to central plane 42 of warming blanket 11. As such, the overall thickness of warming blanket 11 may remain substantially the same dimensionally (e.g. +/−25%) and with respect to central plane 42 over substantially the entirety of upper layer 12 and the bottom layer 13 of the warming blanket.

This feature allows the warming blanket 11 to be deformed and reshaped as shown in FIG. 3A, while the integrity of the passage ways 15 is maintained throughout the warming blanket, This feature may be important as it allows the reshaped warming blanket to be placed over a patient while keeping the bottom layer of the warming blanket proximate to and/or in contact with the patient across the portion of the warming blanket positioned proximate to the patient, thus may provide more efficient warming of the patient. In addition, as shown in FIG. 3A the integrity of the passage ways 15 is maintained throughout the warming blanket, including in the areas between cutouts 23 and 24 where the warming blanket has been deformed to reshape the warming blanket.

In various examples, pleats 54 may be provided along a portion of cutout 23 to allow expansion of upper layer 12, to aid in expanding dimension 30 along the outside curve of periphery 14 within cutout 23. Similar pleats may also be included on bottom layer 13. In addition, pleats 52 may be provided along a portion of cutout 24 to allow folding of upper layer 12, to aid in lessening dimension 32 along the inside curve of periphery 14 within cutout 24. Similar pleats may also be included on bottom layer 13.

FIG. 3B illustrates an example warming system 10 including a forced air warming blanket 11 of FIG. 3A in accordance with one or more example implementations and techniques described in this disclosure. As illustrated, warming blanket 11 is shown in a reshaped configuration as illustrated and described for example with respect to FIG. 3A, now having end 22 rotated approximately 90-degrees relative to the position of end 22 when warming blanket was in the initial configuration. As shown, the portion of warming blanket 11 including the central portion is located over the lower torso of patient 50, with the portion of the warming blanket including end 21 extending over one arm of the patient, and the portion of the warming blanket including end 22 extending over the legs of patient 50. As illustrated in FIG. 3B, axes 33, 34, 35, and 36 are re-oriented from an initial configuration to be oriented in the same orientations as illustrated and described above with respect to FIG. 3A. By reshaping warming blanket 11 as shown in FIG. 3A, the warming blanket 11 can now be applied as shown in FIG. 3B by simply deforming at least the central portion of the warming blanket.

FIG. 4A illustrates a top view of the warming blanket 11 of FIG. 3A, further deformed to reshape the warming blanket into a shape and configuration that is different from the initial shape and configuration, and that is different from the deformed shape and configuration of the warming blanket illustrated in FIG. 3A. As illustrated in FIG. 4A, warming blanket 11 has been deformed so that the dimension indicated by dimension bracket 30 has been stretched to increase this dimension associated the periphery 14 within cutout 23, and the axis 33 along this same portion of periphery 14 has been re-oriented from a straight line to form an arc shape, illustrated as axis 33B. The axis 34 that was originally a straight line along periphery 14 within cutout 24 is also re-oriented, including an arc shape as illustrated by axis 34B, and the dimension associated with dimension bracket 32 is compressed to have a smaller dimension relative to the dimension of dimension bracket 32 relative to this dimension in the initial configuration.

As illustrated in FIG. 4A, axis 35, that originally aligned with end 22 of the warming blanket has been re-oriented approximately 180-degrees, as represented by axis 35B. As illustrated, end 22 aligns with end 21 of warming blanket 11. Similarly, axis 37, which originally aligned with side 36 of warming blanket 11, is re-oriented approximately 180-degrees from the orientation of this axis in the initial configuration, as represented by axis 37B in FIG. 4A. In all instance of re-orientation of axes 33, 34, 35, and 37, the direction and dimension associated with the re-orientation is approximately coplanar or is contained within a plane that is parallel to central plane 42 of warming blanket 11. As such, the overall thickness of warming blanket 11 remains substantially the same dimensionally (+/−10%) and with respect to central plane 42 over substantially the entirety of upper layer 12 and the bottom layer 13 of the warming blanket. Thus, the initial upper body warming blanket was transformed to a lower body blanket.

Again, this feature allows the warming blanket 11 to be deformed and reshaped as shown in FIG. 4A, while remaining substantially flat across the upper and bottom surfaces of the warming blanket. This feature may be important as it allows the reshaped warming blanket to be placed over a patient while keeping the bottom surface of the warming blanket proximate to and/or in contact with the patient across the portion of the warming blanket positioned proximate to the patient, and thus may provide more efficient warming of the patient. This is most easily confirmed by inflating the blanket in the original configuration on a rigid horizontal surface and reshaping the blanket to the second configuration while maintaining the same air flow and pressure. Preferred blankets lay flat on the surface in both configurations. In addition, as shown in FIG. 4A the integrity of the passageways 15 is maintained throughout the warming blanket, including in the areas between cutouts 23 and 24 where the warming blanket has been deformed to reshape the warming blanket.

In various examples, pleats 54 may be provided along a portion of cutout 23 to allow expansion of upper layer 12, to aid in expanding dimension 30 along the outside curve of periphery 14 within cutout 23. Similar pleats may also be included on bottom layer 13. In addition, pleats 52 may be provided along a portion of cutout 24 to allow folding of upper layer 12, to aid in lessening dimension 32 along the inside curve of periphery 14 within cutout 24. Similar pleats may also be included on bottom layer 13. In addition, warming blanket 11 may include one or more secure ties, such as a plastic strip illustrative shown as secure tie 56. Secure tie may be formed, for example, as part of upper layer 12 or as part of bottom layer 13, and extend from one or both of ends 21, 22. When warming blanket 11 has been reshaped as illustrated in FIG. 4A, secure tie 56 may be fastened between ends 21 and 22, for example by being tied to leads form both ends, to helps secure ends 21 and 22 in the position illustrated in FIG. 4A.

FIG. 4B illustrates an example warming system 10 including a forced air warming blanket 11 of FIG. 4A in accordance with one or more example implementations and techniques described in this disclosure. As illustrated, warming blanket 11 is shown in a reshaped configuration as illustrated and described for example with respect to FIG. 4A, now having end 22 rotated approximately 180-degrees relative to the position of end 22 when warming blanket is in the initial configuration. As shown, the portion of warming blanket 11 including the central portion is located over the lower torso, and the portions extending toward ends 21, 22 are positioned to cover both legs of patient 50. As illustrated in FIG. 4B, axes 33B, 34B, 35B, and 36B are re-oriented from an initial configuration to be oriented in the same orientations as illustrated and described above with respect to FIG. 4A. By reshaping warming blanket 11 as shown in FIG. 4A, the warming blanket 11 can now be applied as shown in FIG. 4B by simply deforming at least the central portion of the warming blanket.

FIG. 5 illustrates a top view of an example warming blanket 11 according to the techniques described in this disclosure. As illustrated in FIG. 5, one or more folded pleats, generally indicated by reference number 70, are provided along the periphery 14 of cutout 23. Folded pleats 70 include a fold 72 of the material having upper layer 12 folded over itself, and tacked or otherwise detachably secured along line 71 to the upper layer 12. When secured as shown, folded pleats 70 secure the periphery 14 along and within cutout 23. In order to allow for further expansion of the dimension along periphery 14 within cutout 23, one or more of lines 71 may be detached from the upper layer 12, allowing fold 72 to unfold, and expand the length dimension of periphery 14 within cutout 23. This feature may also be provided on the periphery 14 within cutout 24. Folded pleats 70 allow for deforming and reshaping of a portion of warming blanket 11 without, or in conjunction with, stretching the material or materials used to form the portion of warming blanket that can be deformed to reshape the warming blanket. Additionally, in some embodiments an aluminum wire or other strip or wire of malleable material may be included near the periphery 14 within cutout 24 proximate the lessening dimension 32, to help retain the reshaped warming blanket in re-oriented configuration. Warming blanket 11 as shown in FIG. 5 includes at least one input 16 as described above. The warming blanket 11 as illustrated in FIG. 5 may optionally include an opening 17 according to any of the examples of opening 17 described in this disclosure, and the equivalents thereof.

As also illustrated in FIG. 5, various example of warming blanket 11 may include secure tie 74. Secure tie may be a strip of material that is secured to or within the upper layer 12 near periphery 14 of cutout 24. Secure tie 74 may have a first end 75 at one end of secure tie 74, and a second end 76 at the opposite end of the secure tie, wherein ends 75, 76 may be detachably secured to the external surface of upper layer 12. In various examples, when the warming blanket 11 is being reshaped so that the dimension of periphery 14 within cutout 24 is being reduced, for example as shown in FIG. 3A and FIG. 4A, the ends 75, 76 of secure tie 74 may be drawn together and fastened in a manner that helps compress the periphery 14 and the dimension of the periphery 14 within cutout 24, thus helping maintain warming blanket 11 in the a deformed and reshaped configuration. In various examples, secure tie 74 includes an elastic material configured to contract and help compress the periphery 14 and the dimension of the periphery 14 within cutout 24 when warming blanket 11 is being reshaped in a manner that compresses or tends to reduce the dimension of periphery 14 within cutout 24. One or more of the features and/or functions illustrated and described for the warming blanket 11 as illustrated in FIG. 5 may be incorporated, where appropriate, into any of the examples of warming blankets described in this disclosure.

Examples of warming blankets described herein are not limited to having a particular shape defined by the periphery of the warming blanket as an initial shape and configuration for the warming blanket. Examples of warming blanket 11 provided in FIGS. 1, 2A-2B, 3A-3B, 4A-4B, and 5 are illustrative of a rectangular shaped warming blanket with cutouts that reduce the width dimension over a central portion of the warming blanket. However, other shapes, such as but not limited to rectangular shapes without cutouts, square shapes, and elliptical shapes are examples of other possible shaped for the periphery of a warming blanket and are contemplated by the examples provided in this disclosure.

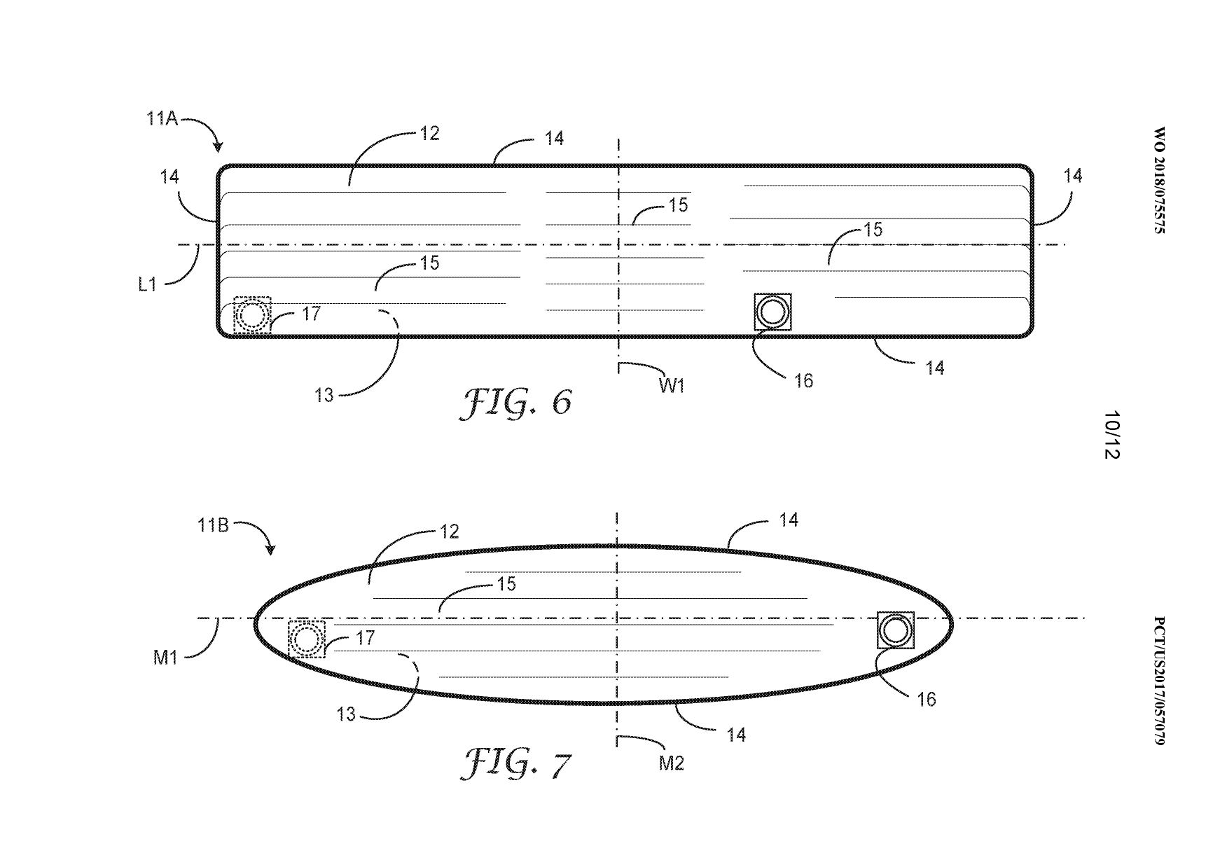

FIG. 6 is an example of a rectangular warming blanket 11A having a porous or non-porous upper layer 12, a porous or perforated bottom layer 13, and a periphery 14 forming an initial shape of a rectangle without cutouts, and including one or more inlets such as an inlet 16 configured to receive an air flow that may be provided to the passageways 15 of the warming blanket. Warming blanket 11A includes a longitudinal axis L1 and a width axis W1. Warming blanket includes at least some portion of the warming blanket that is comprised of material or material that are deformable, either via plastic or elastic deformation, to allow warming blanket 11A to be reshaped so the periphery 14 has a different shape than periphery 14 formed while warming blanket 11A was in the initial configuration. Deformation may include re-orientation of one or both of axes L1 and W1, and wherein after being deformed to reshape warming blanket 11A, the warming blanket remains within an area defined by a thickness dimension of the warming blanket in the initial configuration when reshaped to form the new and different shape and configuration. One or more of the features and/or functions illustrated and described for the warming blanket 11A as illustrated in FIG. 6 may be incorporated, where appropriate, into any of the examples of warming blankets described in this disclosure.

FIG. 7 is an example of an elliptically shaped warming blanket 11B having a non-porous upper layer 12, a porous or perforated bottom layer 13, and a periphery 14 forming an initial configuration having a shape of an ellipse. Warming blanket 11B includes a major axis M1 and a minor axis M2. Warming blanket 11B includes at least some portion of the warming blanket that is comprised of material or material that are deformable, either via plastic or elastic deformation, to allow warming blanket 11B to be reshaped so the periphery 14 has a different shape than formed by periphery 14 while warming blanket 11B was in the initial configuration. Deformation may include re-orientation of one or both of axes M1 and M2, and wherein after being deformed to reshape warming blanket 11B, the warming blanket remains within an area defined by a thickness dimension of the warming blanket in the initial configuration when reshaped to form the new and different shape and configuration. One or more of the features and/or functions illustrated and described for the warming blanket 11B as illustrated in FIG. 7 may be incorporated, where appropriate, into any of the examples of warming blankets described in this disclosure.

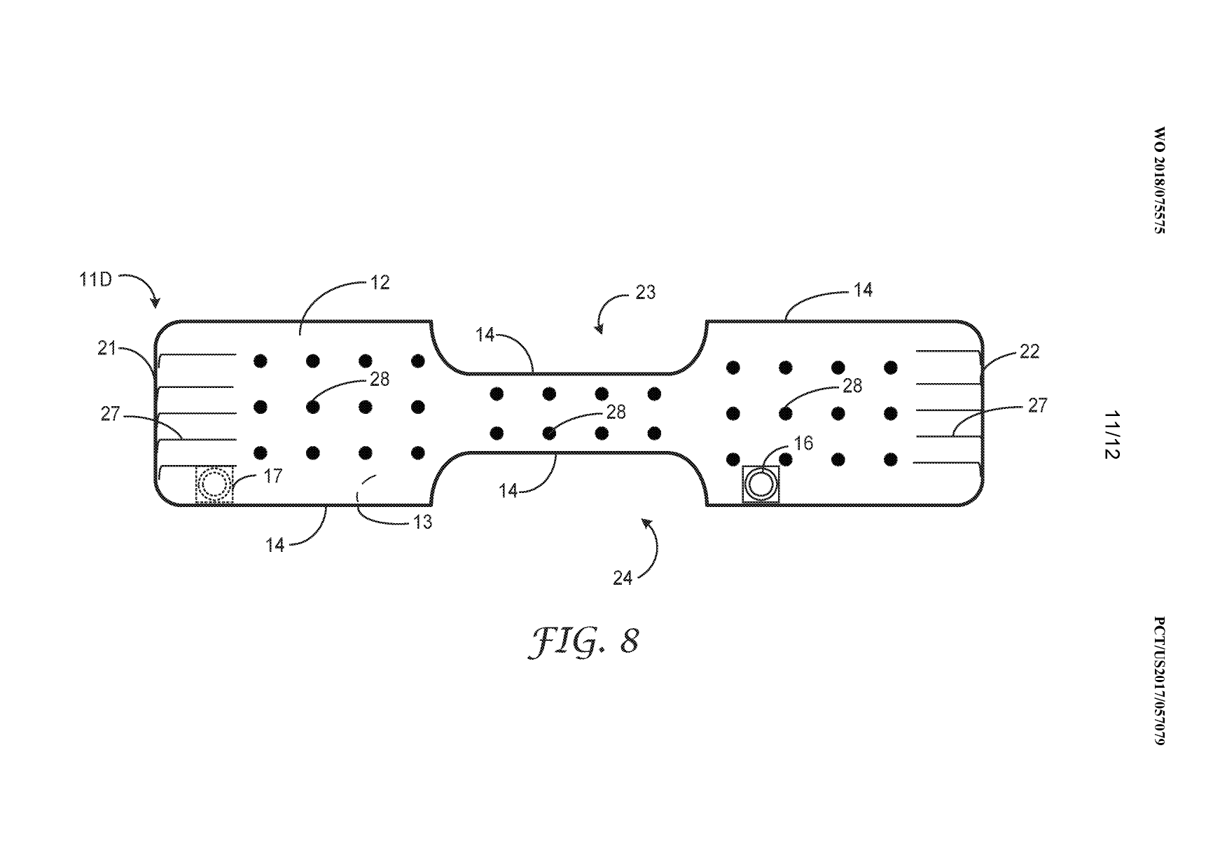

FIG. 8 illustrates a top view of another example warming blanket 11D according to the techniques described in this disclosure. As illustrated in FIG. 8, warming blanket 11D includes top layer 12 and a bottom layer 13 sealing bonded to each other around periphery 14 forming a shape for warming blanket 11D. In addition to being bonded along periphery 14, upper layer 12 and bottom layer 13 are further bonded at various locations within the area enclosed by periphery 14. The further bonding may include linear staking, generally indicated by linear stakes 27, which in some examples may be located at ends 21 and 22 as illustrated in FIG. 8. In addition, at other locations within the areas enclosed by periphery 14, upper layer 12 is bonded to bottom layer 13 by a stake seal, which in some exhales may be formed by a circular area of upper layer 12 being sealing bonded to bottom layer 13 over the circular areas.

The shapes of linear stakes 27 and the shapes of the staked seals are not necessarily limited to the shapes and relative proportions shown in FIG. 8, and may include other shapes, such as wavy lines for linear seals, and for example other shapes for the staked seals, such as a square, triangular, or elliptical shaped area of the upper layer being bonded to the bottom layer to for the staked seal. Further the arrangement, number and relative positioning of the linear seals and the staked seals are not limited to the arrangements as shown in FIG. 8. Linear seals 27 may be position at any location and may have any orientation within the area enclosed by periphery 14, and may or may not have an end of the linear staking that is coupled to a portion of the periphery 14 of the warming blanket. Staked seals 28 are not limited to any particular arrangement of staked seals with respect to location and spacing between the staked seals, and are not necessary limited to being arranged in rows and/or columns as shown in FIG. 8, but may be distributed in other patterns across the area enclose by periphery 14. In some examples, warming blanket 11D only includes staked seals, such as staked seals 28, and does not include linear seals. In other examples, warming blanket 11D includes only linear seals, and does not include staked seals. One or more of the features and/or functions illustrated and described for the warming blanket 11D as illustrated in FIG. 8 may be incorporated, where appropriate, into any of the examples of warming blankets described in this disclosure.

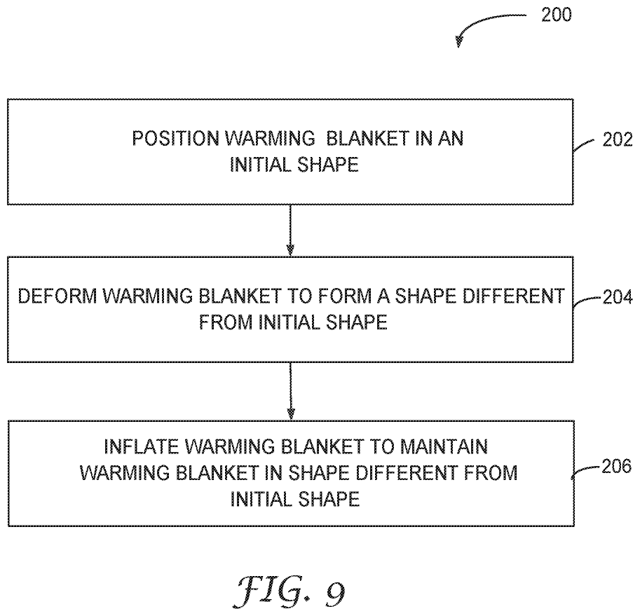

FIG. 9 illustrates a method 200 according to various examples is accordance with the devices, systems, and techniques described in this disclosure. Method 200 is described with respect to system 10 and warming blanket 11 as described for example in FIG. 1 of the disclosure, but is not limited to any particular system or any particular warming blanket, and may be performed with respect to any examples of warming blankets described in this disclosure, and the equivalents thereof. According to method 200, warming blanket 11 is positioned in an initial shape (block 202). The initial shape may be formed with respect to a shape provided by a periphery, such as periphery 14, of the warming blanket. In the initial position, warming blanket may be placed on a substantially flat planar surface, so that the periphery 14 of warming blanket 11 is provided an initial shape having periphery coplanar to the flat planar surface.

According to method 200, the warming blanket incudes at least a portion of the warming blanket that comprises a material or materials that are deformable, either through plastic and/or elastic deformation. Examples of materials that deform include very low density, low density, and linear low density polyolefins, metallocene polyolefins and olefin copolymers such as ethylene-vinyl acetate (EVA). In some examples, the material would be very low density polyethylene optionally containing fillers. An example is Parafilm™ M available from the Bemis Company, Oshkosh Wis. Examples of materials that are elastic are materials that include polyolefins, such as metallocene polyolefins and particularly metallocene polyethylenes such as Engage® polyethylenes (commercially available from Dow Chemical Company, Midland Mich.), polyurethanes such as polyester or polyether polyurethanes (e.g., “Estane® thermoplastic polyurethane,” commercially available from B. F. Goodrich, Cleveland Ohio), polyesters such as polyether polyester (e.g., “Hytrel® polyester elastomer,” commercially available from Du Pont Co., Wilmington, Del.) and plasticized polylactic acid such as Natureworks Ingeo 6202 polylactic acid plasticized with a compatible plasticizer such as a citrate alkyl ester, and polyamides such as polyether polyamides (e.g., “Pebax® Resins” commercially available from ELF Atochem, North America, Inc., Philadelphia, Pa.), acrylic block copolymers such as Kurarity polymers available from Kuraray America Houston, Tex., and styrene block copolymers such as styrene/isoprene/styrene (SIS) and styrene/butadiene/styrene (SBS) available from Kraton Polymers. In order to alter the deformability of these thermoplastics, plasticizers and/or fillers may be added. Preferred plasticizers are soluble and do not migrate out over time. Method 200 includes deforming the warming blanket 11 to form a shape with respect to the periphery 14 that is a different shape from the initial shape formed by the periphery while warming blanket was in the initial shape. Deforming the warming blanket 11 including deforming the warming blanket while maintaining the integrity of the passageways capable of providing distribution of air flows through the interior space and passageways 15 of the warming blanket (block 204). In various examples, deforming the warming blanket includes deforming the shape of the warming blanket so that a thickness dimension of the warming blanket, when inflated, is maintained to about +/−50% of a thickness dimension the warming blanket would assume if inflated while remaining in the initial shape, e.g., the change in thickness dimensions less than 50% across the entire length of the portion of the warming blanket providing passageways 15.

Examples of method 200 include inflating the warming blanket to maintain the warming blanket in the shape that is different from the initial shape (block 206). Maintaining the warming blanket in the shape that is different form the initial shape may include inflating the warming blanket with a flow of air, and placing the warming blanket proximate to a patient to provide warming to the patient while the warming blanket is in the different shape.

The following examples describe one or more aspects of the disclosure.

### Example 1

A warming blanket for warming a patient, the warming blanket comprising: a structure comprising a first layer of material and a second layer of material, the first layer of material forming a bottom layer of the warming blanket, the bottom layer configured to allow a profusion of air through the bottom layer, and the second layer of material forming an upper layer of the warming blanket, the upper layer coupled to the bottom layer around a periphery of the bottom layer to form an initial shape of the warming blanket and to form an interior space between the first layer of material and the second layer of material comprising a plurality of interconnected air passageways; at least one air inlet coupled to the interconnecting air passageways, the inlet configured to receive a flow of air, and to provide the flow of air to the bottom layer through the interconnected air passageways; wherein at least a portion of the structure is configured to be deformable in at least one dimension in order to reshape the periphery of the warming blanket while maintaining the integrity of the interconnecting air passageways throughout the structure, and wherein the portion of the blanket that is deformable is deformable by at least a 50% elongation.

### Example 1a

The warming blanket of example 1, wherein the first layer of material and the second layer of material are both deformable. Example 2. The warming blanket of example 1, wherein the portion of the blanket that is deformable is deformable by at least a 300% elongation.

### Example 2a

The warming blanket of any of the preceding examples, wherein the portion of the blanket that is deformable has a % elongation to force applied ratio of at least 1.25 to 1.

### Example 2b

The warming blanket of any of the preceding examples, wherein the portion of the blanket that is deformable has a % elongation to force applied ratio of at least 30 to 1.

### Example 2c

The warming blanket of any of the preceding examples, wherein the % elongation is measured using ISO 9073-3 (1989).

### Example 2d

The warming blanket of any of the preceding examples, wherein the force applied is Fmax.

### Example 3

The warming blanket of example 1, wherein the portion of the blanket that is deformable is deformable by at least a 350% elongation.

### Example 4

The warming blanket of example 1, wherein the portion of the blanket that is deformable is deformable by at least a 400% elongation.

### Example 5

The warming blanket of example 1, wherein the warming blanket is configured so that when a force of deformation applied is less than 25 Newtons at 25% strain for a test sample of the deformable portion of the blanket that is 2.54 cm wide, according to a tensile strength testing with a gauge length of 50 mm and cross-head speed of 254 mm per minute.

### Example 5a

The warming blanket of example 5, wherein the force of deformation applied is less than 10 Newtons.

### Example 5b

The warming blanket of any of examples 1-5, wherein the first layer of material and the second layer of material is not constructed from a non-woven material.

### Example 6

The warming blanket of any of examples 1-5, wherein the material or materials comprise a low density polyethylene.

### Example 7

The warming blanket of any of examples 1-5, wherein the material or materials comprise a metallocene polyethylene or polypropylene or a styrene block copolymer.

### Example 8

The warming blanket of any of examples 1-5, wherein the material or materials comprise a polyester such as polyether polyester.

### Example 9

The warming blanket of any of examples 1-8, wherein the periphery comprises a rectangular shape having at least one cutout along a side corresponding to a longitudinal axis of the warming blanket.

### Example 10

The warming blanket of example 9, wherein the at last one cutout comprises the portion of the warming blanket that is deformable.

### Example 11

The warming blanket of any of examples 1-10, where the flow of air is maintained at a temperature between 36 to 43 degrees C.

### Example 12

The warming blanket of any of examples 1-11, wherein the structure comprises an end having a width axis aligned with a portion of the periphery forming the end of the structure, the warming blanket configured to be deformable so that the width axis is re-oriented by an amount up to 90-degrees from an initial angle of orientation of the width axis.

### Example 13

The warming blanket of any of examples 1-11, wherein the structure comprises an end having a width axis aligned with a portion of the periphery forming the end of the structure, the warming blanket configured to be deformable so that the width axis is re-oriented by an amount up to 200-degrees from an initial angle of orientation of the width axis.

### Example 14

The warming blanket of any of examples 1-13, wherein the interconnected air passageways are configured to receive a flow of air from the inlet provided in the top layer of the structure, and to distribute the flow of air across the area of the bottom layer in order to provide the profusion of air through the bottom layer.

### Example 15

A system for warming a patient, the system comprising: a source for generating a flow of air; a warming blanket coupled to the source and configured to receive the flow warmed air from the source, and to distribute the flow of air for dispersion to patient, the warming blanket comprising: a structure comprising a first layer of material and a second layer of material, the first layer of material forming a bottom layer of the warming blanket, the bottom layer comprising openings configured to allow a profusion of air through the bottom layer, and the second layer of material forming an upper layer of the warming blanket, the upper layer coupled to the bottom layer around a periphery of the bottom layer to form an initial shape of the warming blanket and to form an interior space between the first layer of material and the second layer of material comprising a plurality of interconnected air passageways; wherein at least a portion of the structure is configured to be deformable in at least one dimension that is co-planer with a central plane of the warming blanket in order to reshape the periphery of the warming blanket having the initial shape so that the structure remains substantially within an area having a thickness dimension of the initial shape of the warming blanket and while maintaining the integrity of the interconnecting air passageways throughout the structure, and wherein the portion of the blanket that is deformable is deformable by at least a 20% elongation.

### Example 16

The system of example 15, wherein the portion of the blanket that is deformable is deformable by at least a 30% elongation.

### Example 17

The system of example 15, wherein the portion of the blanket that is deformable is deformable by at least a 40% elongation.

### Example 18

The system of any of example 15, wherein the portion of the blanket that is deformable is deformable by at least a 50% elongation.

### Example 19

The system of example 15, wherein the warming blanket is configured so that when a force of deformation applied is less than 25 Newtons at 25% strain for a test sample of the deformable portion of the blanket that is 2.54 cm wide, according to a tensile strength testing with a gauge length of 50 mm and cross-head speed of 254 mm per minute.

### Example 20

The system of any of examples 15-19, wherein the flow of air is provided to the warming blanket at a pressure of 100 mm Hg or less.

### Example 21

The system of any of examples 15-20, wherein at least some portion of the structure includes the periphery configured to be deformable by stretching a material or materials comprising that portion of the periphery.

### Example 22

The system of any of examples 15-21, wherein the at least one portion of the structure that is configured to be deformable comprises material or materials comprising a low density polyethylene.

### Example 23

The system of any of examples 15-21, wherein the at least one portion of the structure that is configured to be deformable comprises material or materials comprising a metallocene polyethylene.

### Example 24

The system of any of examples 15-21, wherein the at least one portion of the structure that is configured to be deformable comprises material or materials comprising a polyester such as polyether polyester.

### Example 25

A method of reshaping a warming blanket, the method comprising: positioning the warming blanket to form the warming blanket into an initial shape; deforming the warming blanket to form a shape with respect to the periphery that is a different shape from the initial shape formed by the periphery while maintaining the integrity of the passageways providing distribution of air flows through the interior space of the warming blanket, wherein a portion of the blanket that is deformed is deformed by at least a 20% elongation; and inflating, by the source generating the flow of air, the warming blanket to maintain the warming blanket in the shape that is different from the initial shape.

### Example 26

The method of example 25, wherein the portion of the blanket that is deformed is deformed by at least a 30% elongation.

### Example 27

The method of example 25, wherein the portion of the blanket that is deformed is deformed by at least a 40% elongation.

### Example 28

The method of example 25, wherein the portion of the blanket that is deformed is deformed by at least a 50% elongation.

Various examples of techniques associated with forced-air warming blankets have been described in this disclosure. These and other examples are within the scope of the following claims.

### EXAMPLES

These examples are merely for illustrative purposes only and are not meant to be limiting on the scope of the appended claims. Materials used in the Examples and their sources are provided in Table 1.

TABLE 1
Materials.

| Material | Material Type | Source (Location) |
|---|---|---|
| Bair Hugger Plastic | Polypropylene Film | 3M Corp. (St. Paul, MN) |
| Bair Hugger NW | Polypropylene | 3M Corp. (St. Paul, MN) |
| | Nonwoven | |
| Parafilm | Hydrocarbon Mix | Bemis Company |
| | | (Oshkosh, WI) |
| Steridrape II | Polyethylene Film | 3M Corp. (St. Paul, MN) |
| Tegaderm HP | Polyurethane Film | 3M Corp. (St. Paul, MN) |

### Tensile Testing

Test specimens were prepared and tensile testing was performed according to ISO 9073-3 (1989) with the following parameters at room temperature:
Grip to grip separation at start position: 2.00 in
Speed, start position: 600 mm/min
Test speed: 10 in/min
Force shutdown threshold: 30% Fmax
Force threshold for break investigation: 0.1% Fnom
Travel after break: 5 mm
Speed for travel after break: 10 mm/min
Upper force limit: 200 N.

The tensile testing results provided in Table 2 are the average of at least three replicate tests.

TABLE 2
Results of tensile testing.

| | | | | | | | Elongation | | |
|---|---|---|---|---|---|---|---|---|---|
| | FFₘₐₓ | | Elongation | | F_{break} | St | at break | St | Elongation |
| Material | (N) | St Dev | at Fₘₐₓ (%) | St Dev | (N) | Dev | (%) | Dev | %:Fₘₐₓ |
| Bair Hugger | 8.66 | 0.64 | 304.49 | 32.23 | 6.90 | .35 | 307.30 | 9.68 | 35.5:1 |
| Plastic | | | | | | | | | |
| Bair Hugger | 40.17 | 2.12 | 43.93 | 5.78 | 34.78 | .55 | 51.23 | .05 | 1:1 |
| NW | | | | | | | | | |
| Parafilm | 12.12 | 1.15 | 449.95 | 39.47 | 9.10 | .95 | 473.53 | 1.13 | 37.1:1 |
| Steridrape II | 16.27 | 1.46 | 535.65 | 33.39 | 14.44 | .81 | 543.88 | 6.53 | 32.9:1 |
| Tegaderm HP | 16.06 | 2.66 | 369.65 | 38.77 | 15.72 | .53 | 369.80 | 8.71 | 23:1 |

1. A warming blanket for warming a patient, the warming blanket comprising:
a structure comprising a first layer of material and a second layer of material,
the first layer of material forming a bottom layer of the warming blanket, the bottom layer configured to allow a profusion of air through the bottom layer, and
the second layer of material forming an upper layer of the warming blanket, the upper layer coupled to the bottom layer around a periphery of the bottom layer to form an initial shape of the warming blanket and to form an interior space between the first layer of material and the second layer of material comprising a plurality of interconnected air passageways;
at least one air inlet coupled to the interconnecting air passageways, the inlet configured to receive a flow of air, and to provide the flow of air to the bottom layer through the interconnected air passageways;
wherein at least a portion of the structure is configured to be deformable in at least one dimension in order to reshape the periphery of the warming blanket while maintaining the integrity of the interconnecting air passageways throughout the structure, and wherein the portion of the blanket that is deformable is deformable by at least a 50% elongation.

2. The warming blanket of claim 1, wherein the portion of the blanket that is deformable is deformable by at least a 300% elongation.

3. The warming blanket of claim 1, wherein the portion of the blanket that is deformable is deformable by at least a 350% elongation.

4. The warming blanket of claim 1, wherein the warming blanket is configured so that when a force of deformation applied is less than 25 Newtons at 25% strain for a test sample of the deformable portion of the blanket that is 2.54 cm wide, according to a tensile strength testing with a gauge length of 50 mm and cross-head speed of 254 mm per minute.

5. The warming blanket of claim 1, wherein the first layer of material and second layer of material are both deformable.

6. The warming blanket of claim 1, wherein the material or materials comprise a low density polyethylene.

7. The warming blanket of claim 1, wherein the material or materials comprise a metallocene polyethylene or polypropylene or a styrene block copolymer.

8. The warming blanket of claim 1, wherein the material or materials comprise a polyester such as polyether polyester.

9. The warming blanket of claim 1, wherein the periphery comprises a rectangular shape having at least one cutout along a side corresponding to a longitudinal axis of the warming blanket.

10. The warming blanket of claim 9, wherein the at last one cutout comprises the portion of the warming blanket that is deformable.

11. The warming blanket of claim 1, where the flow of air is maintained at a temperature between 36 to 43 degrees C.

12. The warming blanket of claim 1, wherein the structure comprises an end having a width axis aligned with a portion of the periphery forming the end of the structure, the warming blanket configured to be deformable so that the width axis is re-oriented by an amount up to 90-degrees from an initial angle of orientation of the width axis.

13. The warming blanket of claim 1, wherein the structure comprises an end having a width axis aligned with a portion of the periphery forming the end of the structure, the warming blanket configured to be deformable so that the width axis is re-oriented by an amount up to 200-degrees from an initial angle of orientation of the width axis.

14. The warming blanket of claim 1, wherein the interconnected air passageways are configured to receive a flow of air from the inlet provided in the top layer of the structure, and to distribute the flow of air across the area of the bottom layer in order to provide the profusion of air through the bottom layer.

15. A system for warming a patient, the system comprising:
a source for generating a flow of air;
a warming blanket coupled to the source and configured to receive the flow warmed air from the source, and to distribute the flow of air for dispersion to patient, the warming blanket comprising:
a structure comprising a first layer of material and a second layer of material,
the first layer of material forming a bottom layer of the warming blanket, the bottom layer comprising openings configured to allow a profusion of air through the bottom layer, and
the second layer of material forming an upper layer of the warming blanket, the upper layer coupled to the bottom layer around a periphery of the bottom layer to form an initial shape of the warming blanket and to form an interior space between the first layer of material and the second layer of material comprising a plurality of interconnected air passageways;
wherein at least a portion of the structure is configured to be deformable in at least one dimension that is co-planer with a central plane of the warming blanket in order to reshape the periphery of the warming blanket having the initial shape so that the structure remains substantially within an area having a thickness dimension of the initial shape of the warming blanket and while maintaining the integrity of the interconnecting air passageways throughout the structure, and wherein the portion of the blanket that is deformable is deformable by at least a 50% elongation.

16. The system of claim 15, wherein the portion of the blanket that is deformable is deformable by at least a 300% elongation.

17. The system of claim 15, wherein the portion of the blanket that is deformable is deformable by at least a 350% elongation.

18. The system of claim 15, wherein the warming blanket is configured so that when a force of deformation applied is less than 25 Newtons at 25% strain for a test sample of the deformable portion of the blanket that is 2.54 cm wide, according to a tensile strength testing with a gauge length of 50 mm and cross-head speed of 254 mm per minute.

19. The system of claim 15, wherein the flow of air is provided to the warming blanket at a pressure of 100 mm Hg or less.

20. The system of claim 15, wherein at least some portion of the structure includes the periphery configured to be deformable by stretching a material or materials comprising that portion of the periphery.
